(19) United States
(12) Patent Application Publication
(10) Pub. No.: US 2025/0136925 A1
(43) Pub. Date: May 1, 2025

(54) LACTOBACILLUS PLANTARUM STRAIN WSH048, SCREENING METHOD THEREOF, AND PREPARATION METHOD OF FERMENTED PRODUCT THEREOF

(71) Applicants: Nanjing Ascend Biotechnology Institute Co., Ltd., Jiangsu (CN); NANJING HUASHI NEW MATERIAL CO., LTD, Jiangsu (CN)

(72) Inventors: Luqiu SHI, Jiangsu (CN); Shilan ZHAO, Jiangsu (CN); Hongyu XUE, Jiangsu (CN); Shuai WANG, Jiangsu (CN); Guizhen SU, Jiangsu (CN)

(73) Assignees: Nanjing Ascend Biotechnology Institute Co., Ltd., Jiangsu (CN); NANJING HUASHI NEW MATERIAL CO., LTD, Jiangsu (CN)

(21) Appl. No.: 18/705,595

(22) PCT Filed: Jun. 2, 2022
(86) PCT No.: PCT/CN2022/096891

§ 371 Date: Apr. 29, 2024

(30) Foreign Application Priority Data
Oct. 28, 2021 (CN) 202111266897.9

(51) Int. Cl. C12N 1/20; C12N 1/02; C12P 1/04; C12R 1/25

(52) U.S. Cl. CPC C12N 1/205; C12N 1/02; C12P 1/04; C12N 2500/30; C12N 2500/34; C12N 2500/74; C12N 2500/84; C12R 2001/25

(57) ABSTRACT

Disclosed is a Lactobacillus plantarum strain WSH048, where the Lactobacillus plantarum strain WSH048 has the effects of repairing UV damage, relieving inflammation and preventing skin photoaging. The Lactobacillus plantarum strain WSH048 is Lactobacillus plantarum, with strain No. as WSH048, which was preserved at China General Microbiological Culture Collection Center (CGMCC) on Aug. 20, 2021 with the preservation No. as CGMCC No. 23159. The present disclosure further provides the screening method of Lactobacillus plantarum strain WSH048 and the preparation method of its fermented product. Lactobacillus plantarum strain WSH048 of the present disclosure, having the effects of repairing UV damage, relieving inflammation and preventing skin photoaging, can effectively solve the problems of side effect and expensive price from the current skin inflammation-relieving measures. The screening method for Lactobacillus plantarum strain WSH048 of the present disclosure is characterized by simple steps, being easy to operate and being suitable for promotion.

### TECHNICAL FIELD

The present disclosure belongs to the field of microbial technology, and in particular, relates to a Lactobacillus plantarum strain WSH048, a screening method thereof, and a preparation method of a fermented product thereof.

### BACKGROUND

As the largest organ in human body, human skin is generally divided into epidermal layer, dermis layer and subcutaneous fat layer. The skin can not only act as the primary protective film for living body, but also prevent the water loss in human body and block the penetration of various harmful substances or pollutants externally, thus protecting the skin from UV influence. The skin reduces heat loss by shrinking in cold environment and removes body heat by sweating in hot environment, thus playing an important role in maintaining the stability of human body. However, due to that the skin is frequently and directly exposed to the external environment, it will be subject to many irritations according to the environmental changes and the resulting stress or defense mechanism will lead to a variety of skin diseases.

In 1969, Albert Kligman first proposed that in addition to the inherent aging factors of human body, exposure to sunlight can also cause skin damage and aging. Recently, two epidemiologic studies in Europe and China have provided two populations with different ethnic backgrounds, both of which have revealed a significant correlation between skin aging and exposure to sunlight. The influence of UV radiation on skin has been widely known, and the term “photoaging” also emphasizes the relationship between its cause and effect. According to the wavelength, UV light can be divided into UVA, UVB and UVC, among which UVC cannot reach the earth's surface due to that it is absorbed and dispersed by the atmospheric ozone layer; UVA has strong penetrability to skin, which can affect dermis and subcutaneous tissue areas; UVB has high energy but relatively weak penetrability, mainly affecting skin keratinocytes and forming melanocytes. Long-term UV radiation will not only destroy the outermost skin cells, but also lead to necrosis and metabolic disorder of dermal cells, preventing impurities in the cells from being metabolized and resulting in sunburn, loss of elasticity, premature aging, sensitivity and wrinkles for skin. Moreover, the skin will be subject to prickling, unbearable itching, scratching and bleeding, weakened resistance to diseases, and even edema and exudation after injury, resulting in difficult-to-cure skin diseases such as folliculitis and furuncle. With the aggravation of environmental pollution, the ozone layer is increasingly damaged, the UV light radiated to the earth's surface is gradually increased, and the skin injury by UV light is becoming more and more serious, affecting people's physical and mental health.

Inflammation is a physiological response to protect the body from harmful environmental factors, such as the invasion of bacteria and other foreign objects. Inflammation may cause a significant increase in several polymorphonuclear neutrophils (PMN) and immune substances, which can heal and protect the skin by secreting inflammatory cell products such as various proteolytic enzymes and cytokinins. However, the elastase, hyaluronidase, lipoxygenase and lipolytic enzymes produced during inflammation may sometimes cause damage or even harm to adjacent tissue cells and non-cellular components. Therefore, inflammation may facilitate the functional recovery under appropriate conditions. When the substances spurring inflammation do not disappear or continue to be produced, it will cause chronic inflammation, resulting in more serious damage to tissues.

In terms of the sunscreen cosmetics utilizing the existing technology, most of them are focus on UV protection, such as some sunscreen products with high SFP value and PA. As we know, skin is the first physiological barrier for the body to contact with external environment. Although these broad-spectrum sunscreen cosmetics can protect the skin from UV damage to a great extent, they can't repair the sunburned skin. At present, chemical exfoliation, photoelectric repair (intense pulsed light, fractional laser, near-infrared light, etc.) and injection reshaping (botulinum toxin and hyaluronic acid injection, autologous fat filling) are mostly used in clinical practice to relieve the skin photoaging. Chemical exfoliation and injection reshaping may lead to skin swelling, itching and other symptoms, and are accompanied by risks. Although photoelectric repair has small side effects, its expensive price limits the widespread application. Probiotics have been recognized by medical and scientific circles in recent years as beneficial to human health and no side effects. Therefore, it is necessary to provide a probiotic that can repair UV damage on skin, relieve inflammation and prevent skin photoaging.

### SUMMARY OF INVENTION

To solve these problems, the present disclosure provides a Lactobacillus plantarum strain WSH048 and screening method thereof as well as the preparation method of its fermented product. Lactobacillus plantarum strain WSH048 of the present disclosure, having the effects of repairing UV damage, relieving inflammation and preventing skin photoaging, can effectively solve the problems of side effect and expensive price from the current skin inflammation-relieving measures.

The objective of the present disclosure and the solutions to its technical problems are achieved through the following technical proposal.

According to an aspect of the present disclosure, a Lactobacillus plantarum strain WSH048 is provided. The Lactobacillus plantarum strain WSH048 has the effects of repairing UV damage, relieving inflammation and preventing skin photoaging. The Lactobacillus plantarum strain WSH048 is Lactobacillus plantarum, with strain No. as WSH048, which was preserved at China General Microbiological Culture Collection Center (CGMCC) on Aug. 20, 2021 with the preservation No. as CGMCC No. 23159.

The objective of the present disclosure and the solutions to its technical problems are further achieved through the following technical proposal.

According to a further aspect of the present disclosure, a method for screening Lactobacillus plantarum strain WSH048 is provided. The method comprises the following steps:
S1: Collect the samples, and culture the samples at 37° C. for 48 h,
S2: Furnish selective solid culture medium;
S3: Pour the above-mentioned selective solid culture medium into a plate until solidification, smear the cultured samples on the selective solid culture medium, put it in an incubator for culture, select the strains from the colony with calcium dissolving zone on the plate, perform the gene sequence alignment and determine the strain category, thus screening Lactobacillus plantarum strain WSH048.

Preferably, the samples in step SI are collected from yogurt.
preferably, the selective solid culture medium in step S2 is prepared as per the following method: Adding calcium percarbonate solution to the sterilized MRS culture medium to make the concentration of calcium carbonate in the culture medium become 2%, and thus obtaining the selective solid culture medium.

Preferably, the MRS culture medium is prepared with peptone 1%, beef powder 0.5%, glucose 2%, yeast powder 0.4%, sodium acetate 0.5%, dipotassium hydrogen phosphate 0.2%, ammonium dihydrogen citrate 0.2%, Tween-80 0.1%, magnesium sulfate 0.02%, manganese sulfate 0.005% and agar powder 2% by adjusting pH to 6.3, adding distilled water to 1000 mL and sterilizing at 115° C. for 20 min.

Preferably, the culture conditions in step S3 is as: Temperature 37° C. and duration 32 h.

The objective of the present disclosure and the solutions to its technical problems are further achieved through the following technical proposal.

According to a further aspect of the present disclosure, a method for preparing the fermented product of Lactobacillus plantarum strain WSH048 is provided. The method comprises the following steps:
(1) Inoculate Lactobacillus plantarum strain WSH048 to MRS solid culture medium and perform activated culture at 37° C. for 1 day to obtain activated Lactobacillus plantarum strain WSH048. After that, inoculate the activated Lactobacillus plantarum strain WSH048 to MRS liquid culture medium and perform culture at 37° C. for 1 day to obtain the bacteria solution of Lactobacillus plantarum strain WSH048;
(2) Inoculate the bacteria solution of Lactobacillus plantarum strain WSH048 to the fermented solution as per inoculation dosage of 10% to obtain the fermented solution of Lactobacillus plantarum strain WSH048;
(3) Centrifuge the fermented solution of Lactobacillus plantarum strain WSH048 (30 OD/mL), collect, wash and resuspend the bacterial cells, and finally perform pasteurization to obtain the fermented product.

Preferably, the fermented solution in step (2) is prepared with peptone 1%, beef powder 0.5%, glucose 2%, yeast powder 0.4%, sodium acetate 0.5%, dipotassium hydrogen phosphate 0.2%, ammonium dihydrogen citrate 0.2%, Tween-80 0.1%, magnesium sulfate 0.02% and manganese sulfate 0.005% by adjusting pH to 6.3, adding distilled water to 1000 mL and sterilizing at 115° C. for 20 min.

Preferably, the fermentation conditions in step (2) is as: Temperature 30-37° C. and duration 30-50 h.

Preferably, the centrifugation conditions in step (3) is as: Rotating speed 6000 rmp and duration 20 min.

Preferably, the 0.9% normal saline in step (3) is adopted for washing and resuspending the bacterial cells.

With the above technical proposals, the present disclosure has at least the following advantages: Lactobacillus plantarum strain WSH048 of the present disclosure, having the effects of repairing UV damage, relieving inflammation and preventing skin photoaging, can effectively solve the problems of side effect and expensive price from the current skin inflammation-relieving measures. The screening method for Lactobacillus plantarum strain WSH048 of the present disclosure is characterized by simple steps, being easy to operate and being suitable for promotion.

The foregoing description is only an overview of the technical proposals of the present disclosure. In order to make the technical means of the present disclosure more clearly understood and implemented in accordance with the contents of the description, the following detailed description is given with reference to the preferred embodiments and drawings of the present disclosure.

### BRIEF DESCRIPTION OF DRAWINGS

FIG. 1 is a RGR (relative growth rate) result diagram for HDF cells of Lactobacillus plantarum strain WSH048 according to Embodiment 2;

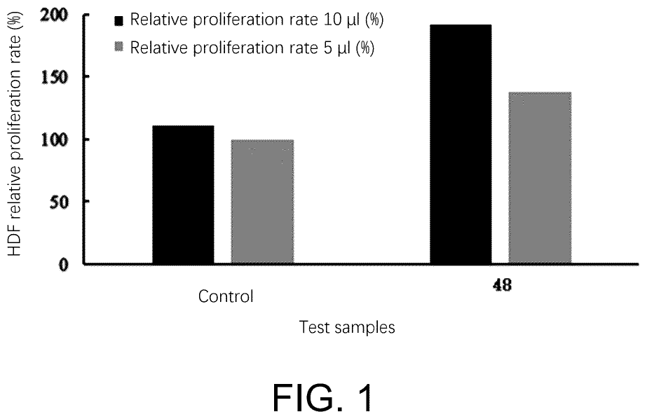

FIG. 2 is a Q-PCR result diagram for IL-6 cells of Lactobacillus plantarum strain WSH048 according to Embodiment 2.

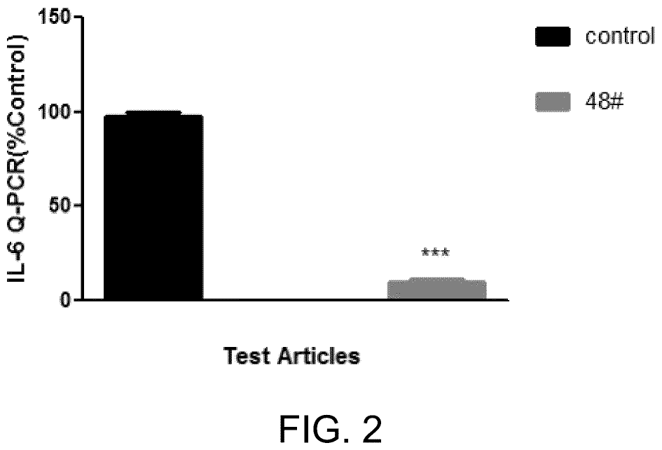

### DESCRIPTION OF EMBODIMENTS

In order to make the technical means, the creation features, the achievement purposes and the effects of the present disclosure easy to understand, the technical proposals in the embodiments of the present disclosure will be clearly and completely described below with reference to the embodiments and drawings of the present disclosure, and it is obvious that the described embodiments are only a part but not all of the embodiments of the present disclosure. All other embodiments, which can be derived by those skilled in the art from the embodiments given herein without making any creative effort, shall fall within the protection scope of the present disclosure.

### Example 1

### 1. Screening for Lactobacillus Plantarum

(1) Collect yogurt samples and culture them in an incubator for 48 h.
(2) Add calcium percarbonate solution to the sterilized MRS culture medium with the concentration of calcium carbonate in the culture medium as 2%, and thus obtain the selective solid culture medium, among which MRS culture medium is prepared with peptone 1%, beef powder 0.5%, glucose 2%, yeast powder 0.4%, sodium acetate 0.5%, dipotassium hydrogen phosphate 0.2%, ammonium dihydrogen citrate 0.2%, Tween-80 0.1%, magnesium sulfate 0.02%, manganese sulfate 0.005% and agar powder 2% by adjusting pH to 6.3, adding distilled water to 1000 mL and sterilizing at 115° C. for 20 min.
(3) Pour the selective solid culture medium in step (2) into a plate and mix it evenly until solidification, take yogurt from the incubator and smear it on the culture medium, put it in an incubator at 37° C. to have 32 h stationary culture, select the strains from the colony with calcium dissolving zone on the plate, perform the gene sequence alignment and determine the strain category, thus screening Lactobacillus plantarum strain WSH048.
(4) Perform gradient dilution test after sterile preparation of Lactobacillus plantarum, and perform MTT cell activity test with HDF (human dermal fibroblast). Evaluate and test the inhibition by the samples on cell proliferation through the absorbance detected by ELISA instrument, and select the Lactobacillus plantarum with the best inflammatory cytokine-inhibiting effect for culture medium optimization and fermentation in a fermentation tank.

### 2. Preparation for the Fermented Product of Lactobacillus Plantarum Strain WSH048

(1) Inoculate Lactobacillus plantarum strain WSH048 to MRS solid culture medium and perform activated culture at 37° C. for 1 day to obtain activated Lactobacillus plantarum strain WSH048. After that, inoculate the activated Lactobacillus plantarum strain WSH048 to MRS liquid culture medium and perform culture at 37° C. for 1 day to obtain the bacteria solution of Lactobacillus plantarum strain WSH048.
(2) Inoculate the bacteria solution of Lactobacillus plantarum strain WSH048 to the fermented solution with inoculation dosage of 10% and ferment it at 30° C. for 50 h to obtain the fermented solution of Lactobacillus plantarum strain WSH048, among which the fermented solution is prepared with peptone 1%, beef powder 0.5%, glucose 2%, yeast powder 0.4%, sodium acetate 0.5%, dipotassium hydrogen phosphate 0.2%, ammonium dihydrogen citrate 0.2%, Tween-80 0.1%, magnesium sulfate 0.02% and manganese sulfate 0.005% by adjusting pH to 6.3, adding distilled water to 1000 mL and sterilizing at 115° C. for 20 min.
(3) Centrifuge the fermented solution of Lactobacillus plantarum strain WSH048 (30OD/mL) at 6000 rpm for 20 min, collect, wash and resuspend the bacterial cells with 0.9% normal saline, and finally perform pasteurization.

### 3. Functional Test for Skin Repair

### (1) Cell Culturing and Passaging

When the cell's confluence degree reaches 90%, the cells can be passaged, washed with PBS for 1-2 times after removing the supernatant, dissociated with appropriate amount of trypsin for about 8 min, and then added with complete medium to stop dissociation. At this time, the cells can be passaged as 1:3-1:6, and cultured in an incubator with 5% CO₂ at 37° C. after being passaged.

### (2) Cell Seeding

After the cell state and confluence rate are observed, the cells are seeded, centrifuged after dissociation, stained with trypan blue and counted under a microscope. The well-seeded cell culture plates are placed in a incubator for 24 h stationary culture.

### (3) Test Samples Adding

Observe whether the cell density reaches the confluence degree required by the experiment, add the test samples, add 10 ul from the first well and then make two-fold dilution, with the last well as negative control. After the samples are added, continue to have 24 h stationary culture in a incubator.

### (4) Chemical Dosing and Testing

After 10 ul MTT is added to each well, have 4 h light-tight stationary culture in a incubator, remove the supernatant, add 150 ul DMSO solvent to each well, and shake it on a horizontal shaker for 10 min for full dissolution. After dissolution, the absorbance of each well is measured at OD490 nm of an ELISA instrument. Among them, the inflammatory cytokine-inhibiting rate of Lactobacillus plantarum strain WSH048 reaches 90%.

### Example 2

### 1. Screening for Lactobacillus Plantarum

(1) Collect yogurt samples and culture them in an incubator for 48 h.
(2) Add calcium percarbonate solution to the sterilized MRS culture medium with the concentration of calcium carbonate in the culture medium as 2%, and thus obtain the selective solid culture medium, among which MRS culture medium is prepared with peptone 1%, beef powder 0.5%, glucose 2%, yeast powder 0.4%, sodium acetate 0.5%, dipotassium hydrogen phosphate 0.2%, ammonium dihydrogen citrate 0.2%, Tween-80 0.1%, magnesium sulfate 0.02%, manganese sulfate 0.005% and agar powder 2% by adjusting pH to 6.3, adding distilled water to 1000 mL and sterilizing at 115° C. for 20 min.
(3) Pour the selective solid culture medium in step (2) into a plate and mix it evenly until solidification, take yogurt from the incubator and smear it on the culture medium, put it in an incubator at 37° C. to have 32 h stationary culture, select the strains from the colony with calcium dissolving zone on the plate, perform the gene sequence alignment and determine the strain category, thus screening Lactobacillus plantarum strain WSH048.
(4) Perform gradient dilution test after sterile preparation of Lactobacillus plantarum, and perform MTT cell activity test with HDF (human dermal fibroblast). Evaluate and test the inhibition by the samples on cell proliferation through the absorbance detected by ELISA instrument, and select the Lactobacillus plantarum with the best inflammatory cytokine-inhibiting effect for culture medium optimization and fermentation in a fermentation tank.

### 2. Preparation for the Fermented Product of Lactobacillus Plantarum Strain WSH048

(1) Inoculate Lactobacillus plantarum strain WSH048 to MRS solid culture medium and perform activated culture at 37° C. for 1 day to obtain activated Lactobacillus plantarum strain WSH048. After that, inoculate the activated Lactobacillus plantarum strain WSH048 to MRS liquid culture medium and perform culture at 37° C. for 1 day to obtain the bacteria solution of Lactobacillus plantarum strain WSH048.
(2) Inoculate the bacteria solution of Lactobacillus plantarum strain WSH048 to the fermented solution with inoculation dosage of 10% and ferment it at 34° C. for 36 h to obtain the fermented solution of Lactobacillus plantarum strain WSH048, among which the fermented solution is prepared with peptone 1%, beef powder 0.5%, glucose 2%, yeast powder 0.4%, sodium acetate 0.5%, dipotassium hydrogen phosphate 0.2%, ammonium dihydrogen citrate 0.2%, Tween-80 0.1%, magnesium sulfate 0.02% and manganese sulfate 0.005% by adjusting pH to 6.3, adding distilled water to 1000 mL and sterilizing at 115° C. for 20 min.
(3) Centrifuge the fermented solution of Lactobacillus plantarum strain WSH048 (30 OD/mL) at 6000 rpm for 20 min, collect, wash and resuspend the bacterial cells with 0.9% normal saline, and finally perform pasteurization.

### 3. Functional Test for Skin Repair

### (1) Cell Culturing and Passaging

When the cell's confluence degree reaches 90%, the cells can be passaged, washed with PBS for 1-2 times after removing the supernatant, dissociated with appropriate amount of trypsin for about 8 min, and then added with complete medium to stop dissociation. At this time, the cells can be passaged as 1:3-1:6, and cultured in an incubator with 5% CO₂ at 37° C. after being passaged.

### (2) Cell Seeding

After the cell state and confluence rate are observed, the cells are seeded, centrifuged after dissociation, stained with trypan blue and counted under a microscope. The well-seeded cell culture plates are placed in an incubator for 24 h stationary culture.

### (3) Test Samples Adding

Observe whether the cell density reaches the confluence degree required by the experiment, add the test samples, add 10 ul from the first well and then make two-fold dilution, with the last well as negative control. After the samples are added, continue to have 24 h stationary culture in a incubator.

### (4) Chemical Dosing and Testing

After 10 ul MTT is added to each well, have 4 h light-tight stationary culture in a incubator, remove the supernatant, add 150 ul DMSO solvent to each well, and shake it on a horizontal shaker for 10 min for full dissolution. After dissolution, the absorbance of each well is measured at OD490 nm of an ELISA instrument. Among them, the inflammatory cytokine-inhibiting rate of Lactobacillus plantarum strain WSH048 reaches 90%.

### Example 3

### 1. Screening for Lactobacillus Plantarum

(1) Collect yogurt samples and culture them in an incubator for 48 h.
(2) Add calcium percarbonate solution to the sterilized MRS culture medium with the concentration of calcium carbonate in the culture medium as 2%, and thus obtain the selective solid culture medium, among which MRS culture medium is prepared with peptone 1%, beef powder 0.5%, glucose 2%, yeast powder 0.4%, sodium acetate 0.5%, dipotassium hydrogen phosphate 0.2%, ammonium dihydrogen citrate 0.2%, Tween-80 0.1%, magnesium sulfate 0.02%, manganese sulfate 0.005% and agar powder 2% by adjusting pH to 6.3, adding distilled water to 1000 mL and sterilizing at 115° C. for 20 min.
(3) Pour the culture medium in step (2) into a plate and mix it evenly until solidification, take yogurt from the incubator and smear it on the culture medium, put it in an incubator at 37° C. to have 32 h stationary culture, select the strains from the colony with calcium dissolving zone on the plate, perform the gene sequence alignment and determine the strain category, thus screening Lactobacillus plantarum strain WSH048.
(4) Perform gradient dilution test after sterile preparation of Lactobacillus plantarum, and perform MTT cell activity test with HDF (human dermal fibroblast). Evaluate and test the inhibition by the samples on cell proliferation through the absorbance detected by ELISA instrument, and select the Lactobacillus plantarum with the best inflammatory cytokine-inhibiting effect for culture medium optimization and fermentation in a fermentation tank.

### 2. Preparation for the Fermented Product of Lactobacillus Plantarum Strain WSH048

(1) Inoculate Lactobacillus plantarum strain WSH048 to MRS solid culture medium and perform activated culture at 37° C. for 1 day to obtain activated Lactobacillus plantarum strain WSH048. After that, inoculate the activated Lactobacillus plantarum strain WSH048 to MRS liquid culture medium and perform culture at 37° C. for 1 day to obtain the bacteria solution of Lactobacillus plantarum strain WSH048.
(2) Inoculate the bacteria solution of Lactobacillus plantarum strain WSH048 to the fermented solution with inoculation dosage of 10% and ferment it at 37° C. for 48 h to obtain the fermented solution of Lactobacillus plantarum strain WSH048, among which the fermented solution is prepared with peptone 1%, beef powder 0.5%, glucose 2%, yeast powder 0.4%, sodium acetate 0.5%, dipotassium hydrogen phosphate 0.2%, ammonium dihydrogen citrate 0.2%, Tween-80 0.1%, magnesium sulfate 0.02% and manganese sulfate 0.005% by adjusting pH to 6.3, adding distilled water to 1000 mL and sterilizing at 115° C. for 20 min.
(3) Centrifuge the fermented solution of Lactobacillus plantarum strain WSH048 (30 OD/mL) at 6000 rpm for 20 min, collect, wash and resuspend the bacterial cells with 0.9% normal saline, and finally perform pasteurization.

### 3. Functional Test for Skin Repair

### (1) Cell Culturing and Passaging

When the cell's confluence degree reaches 90%, the cells can be passaged, washed with PBS for 1-2 times after removing the supernatant, dissociated with appropriate amount of trypsin for about 8 min, and then added with complete medium to stop dissociation. At this time, the cells can be passaged as 1:3-1:6, and cultured in an incubator with 5% CO₂ at 37° C. after being passaged.

### (2) Cell Seeding

After the cell state and confluence rate are observed, the cells are seeded, centrifuged after dissociation, stained with trypan blue and counted under a microscope. The well-seeded cell culture plates are placed in a incubator for 24 h stationary culture.

### (3) Test Samples Adding

Observe whether the cell density reaches the confluence degree required by the experiment, add the test samples, add 10 ul from the first well and then make two-fold dilution, with the last well as negative control. After the samples are added, continue to have 24 h stationary culture in a incubator.

### (4) Chemical Dosing and Testing

After 10 ul MTT is added to each well, have 4 h light-tight stationary culture in a incubator, remove the supernatant, add 150 ul DMSO solvent to each well, and shake it on a horizontal shaker for 10 min for full dissolution. After dissolution, the absorbance of each well is measured at OD490 nm of an ELISA instrument. Among them, the inflammatory cytokine-inhibiting rate of Lactobacillus plantarum strain WSH048 reaches 90%.

The above is only a preferred embodiment of the present disclosure, and is not intended to limit the scope of the present disclosure. Although the present disclosure has been disclosed in the above preferred embodiments, it is not intended to limit the present disclosure. Those skilled in the art can make some modifications or modifications to the equivalent embodiments by using the above-disclosed technical contents without departing from the technical scope of the present disclosure, but without departing from the technical solution of the present disclosure, according to the present disclosure. Any modification, equivalent change and modification of the above embodiments according to the technical substantials of the present disclosure are still within the scope of the technical solution of the present disclosure.

What is claimed is:

1. A Lactobacillus plantarum strain WSH048, wherein the Lactobacillus plantarum strain WSH048 has effects of repairing ultraviolet damage, relieving inflammation, and preventing skin photoaging, and the Lactobacillus plantarum strain WSH048 is Lactobacillus plantarum with a strain number of WSH048, and is deposited in China General Microbiological Culture Collection Center (CGMCC) on Aug. 20, 2021 with an accession number of CGMCC No. 23159.

2. A method for screening the Lactobacillus plantarum strain WSH048 according to claim 1, comprising the following steps:
step S1: collecting samples, and culturing the samples at 37° C. for 48 hours to obtain cultured samples;
step S2: preparing a selective solid culture medium; and
step S3: pouring the selective solid culture medium into a plate, after the selective solid culture medium is solidified, spreading the cultured samples on the selective solid culture medium, placing the selective solid culture medium with the cultured samples in an incubator for cultivation, picking a strain from a colony with a calcium dissolving zone on the plate, and subjecting the strain to a gene sequence alignment to determine a species of the strain, to screen out the Lactobacillus plantarum strain WSH048.

3. The method according to claim 2, wherein the samples in the step SI are collected from a yogurt.

4. The method according to claim 2, wherein the selective solid culture medium in the step S2 is prepared by adding a calcium percarbonate solution to a sterilized De Man-Rogosa-Sharpe (MRS) culture medium to allow a concentration of calcium carbonate in the MRS culture medium to be 2% to obtain the selective solid culture medium.

5. The method according to claim 4, wherein the MRS culture medium is prepared with 1% peptone, 0.5% beef powder, 2% glucose, 0.4% yeast powder, 0.5% sodium acetate, 0.2% dipotassium phosphate, 0.2% ammonium dihydrogen citrate, 0.1% Tween-80, 0.02% magnesium sulfate, 0.005% manganese sulfate, and 2% agar powder by adjusting a pH value to 6.3, adding distilled water to 1,000 mL, followed by sterilizing at 115° C. for 20 minutes.

6. The method according to claim 2, wherein the cultivation in the step S3 is performed for 32 hours at 37° C.

7. A method for preparing a fermented product of the Lactobacillus plantarum strain WSH048 according to claim 1, comprising the following steps:
step (1) inoculating the Lactobacillus plantarum strain WSH048 to a MRS solid culture medium for activation and cultivation at 37° C. for 1 day to obtain an activated Lactobacillus plantarum strain WSH048, then inoculating the activated Lactobacillus plantarum strain WSH048 to a MRS liquid culture medium for cultivation at 37° C. for 1 day to obtain a bacterial suspension of the Lactobacillus plantarum strain WSH048;
step (2) inoculating the bacterial suspension of the Lactobacillus plantarum strain WSH048 to a broth at an inoculum size of 10% for a fermentation, to obtain a fermentation broth of the Lactobacillus plantarum strain WSH048; and
step (3) centrifuging the fermentation broth of the Lactobacillus plantarum strain WSH048 at 30 OD/mL, collecting bacterial cells, washing and resuspending the bacterial cells, followed by pasteurization to obtain the fermented product.

8. The method according to claim 7, wherein the broth in the step (2) is prepared with 1% peptone, 0.5% beef powder, 2% glucose, 0.4% yeast powder, 0.5% sodium acetate, 0.2% dipotassium phosphate, 0.2% ammonium dihydrogen citrate, 0.1% Tween-80, 0.02% magnesium sulfate, and 0.005% manganese sulfate by adjusting a pH value to 6.3, adding distilled water to 1,000 mL, followed by sterilizing at 115° C. for 20 minutes.

9. The method according to claim 7, wherein the fermentation in the step (2) is performed for 30 hours to 50 hours at 30° C. to 37° C.

10. The method according to claim 7, wherein in the step (3), the fermentation broth of the Lactobacillus plantarum strain WSH048 is centrifuged at a rotation speed of 6,000 rmp for 20 minutes.
